(19) United States
(12) Patent Application Publication
(10) Pub. No.: US 2025/0041420 A1
(43) Pub. Date: Feb. 6, 2025

(54) NANOROBOT DESIGN, FUNCTION, AND FABRICATION PROCESS

(71) Applicant: Andrew Robert Haverly, Kirkland, WA (US)

(72) Inventor: Andrew Robert Haverly, Kirkland, WA (US)

(73) Assignee: Andrew Robert Haverly, Kirkland, WA (US)

(21) Appl. No.: 18/793,823

(22) Filed: Aug. 4, 2024

Related U.S. Application Data
(63) Continuation-in-part of application No. 63/517,901, filed on Aug. 5, 2023.
(63) Continuation-in-part of application No. 63/517,902, filed on Aug. 5, 2023.

(51) Int. Cl. A61K 41/00; A61K 9/00; B82Y 15/00; G01N 27/414

(52) U.S. Cl. CPC A61K 41/0052; A61K 9/0009; G01N 27/4146; B82Y 15/00

(57) ABSTRACT

The present invention is a system of nanorobots comprising; a fabrication method, a power generator; electrically controlled proteins; sensors; a method of killing an organism's cells; and a method of communicating with the nanorobots with an outside device; wherein a nanorobot is fabricated, put into the organism, powered, uses sensors to determine whether to kill the organism's cells, selectively kills the organism's cells, the nanorobots can communicate with an outside device, and the nanorobots can release drugs.

### CROSS-REFERENCE TO RELATED APPLICATIONS

This application is a continuation-in-part (and claims the benefit of priority under 35 USC 120) of U.S. application 63/517,901 filed Aug. 5, 2023 currently pending and U.S. application No. 63/517,902 filed Aug. 5, 2023.

### BACKGROUND OF THE INVENTION

The present invention relates to nano-scale and micro-scale robots, their manufacture, and their function.

Nano-scale and micro-scale robots (hereinafter both are considered nanorobots) are computers and robots that range in size from 1 nm to 1000 μm.

Nanorobots can have many uses, but the primary ones are for medicine.

Nanorobots are hard to manufacture and nanorobots have difficulty interacting with the world as macro-scale and nano-scale motors are fundamentally different.

Nanorobots are programmable or functionally designed devices. They can perceive their environment and act accordingly and they can potentially be used to cure diseases like cancers and infections.

Thin film transistors (TFT) are transistors that are fabricated on a nonreactive surface (substrate). These TFT are most frequently used in scenarios where they are also on a transparent surface. TFT can be used to create nanorobots with only some small changes in the typical TFT fabrication scenarios.

Cells are sensitive to heat. If they get too hot, they die. This is true for all cells, including cancer cells and bacteria. Selectively heating cancer cells and not other cells can allow for targeted cancer treatment. The same can be done for infectious cells and viruses.

The current state of the art for nanorobots and their manufacture is more focused on biology/chemistry and less focused on their digital implementation. There are many advances in the biochemical nanorobots that can be integrated in this invention's design, but there is relatively little focus on using actual transistors in nanorobots.

The most advanced integrated circuit nanorobot designs publicly available are those of the article: https://doi.org/10.1038/s41565-018-0194-z. Compared to the present invention, these nanoparticles are much simpler, are much larger due to the fabrication technique, are two dimensional, and do not have many of the capabilities described herein.

Nanoelectromechanical and microelectromechanical systems are systems in which a nanorobot can control motor-like systems.

It is desired to have novel approaches to nanorobot manufacturing and nanorobot capabilities.

### SUMMARY

Accordingly, in a first embodiment, the present invention is a system of nanorobots comprising; a fabrication method, a power generator, electrically controlled proteins, sensors, and a method of killing an organism's cells; wherein a nanorobot is fabricated, put into the organism, powered, uses sensors to determine whether to kill the organism's cells, and selectively kills the organism's cells.

Accordingly, in a second embodiment, the present invention is a system of nanorobots comprising; a fabrication method, a power generator, electrically controlled proteins, sensors, a method of killing an organism's cells, and a method of communicating with the nanorobots with an outside device; wherein a nanorobot is fabricated, put into the organism, powered, uses sensors to determine whether to kill the organism's cells, selectively kills the organism's cells, and the nanorobots can communicate with an outside device.

### BRIEF DESCRIPTION OF THE DRAWINGS

FIG. 1 depicts an illustration of a nanorobotics fabrication process, in accordance with one embodiment of the present invention.

FIG. 2 depicts an illustration of a nanorobotics fabrication process, in accordance with one embodiment of the present invention.

FIG. 3 depicts an illustration of a nanorobotics fabrication process, in accordance with one embodiment of the present invention.

FIG. 4 depicts an illustration of a nanorobotics system design, in accordance with one embodiment of the present invention.

FIG. 5 depicts an illustration of a nanorobot, in accordance with one embodiment of the present invention.

FIG. 6 depicts an illustration of a nanorobot, in accordance with one embodiment of the present invention.

FIG. 7 depicts an illustration of a nanorobotics system, in accordance with one embodiment of the present invention.

FIG. 8 depicts an illustration of a nanorobot, in accordance with one embodiment of the present invention.

FIG. 9 depicts an illustration of a nanorobot, in accordance with one embodiment of the present invention.

### DETAILED DESCRIPTION OF THE INVENTION

The present invention provides a method and a system that allows for the biointegration of nanorobots through the use of a microscopic integrated circuit and various embodiments of systems to achieve the increased presence of computationally capable devices in the body to advance medicine. This method and system is used, in some instances, to kill cancerous cells.

This is advantageous for a number of reasons, in that the method allows for more complex operations within the body, it allows for many more operations than previously capable, it allows for increased precision in medicine, and it allows for more precise biointegration.

Through the use of the method and system described herein, the precise medical treatment described herein, through the manufacture, design, and biointegration of nanorobots can cure diseases from the process described below.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely”, “only” and the like in connection with the recitation of claim elements or use of a “negative” limitation.

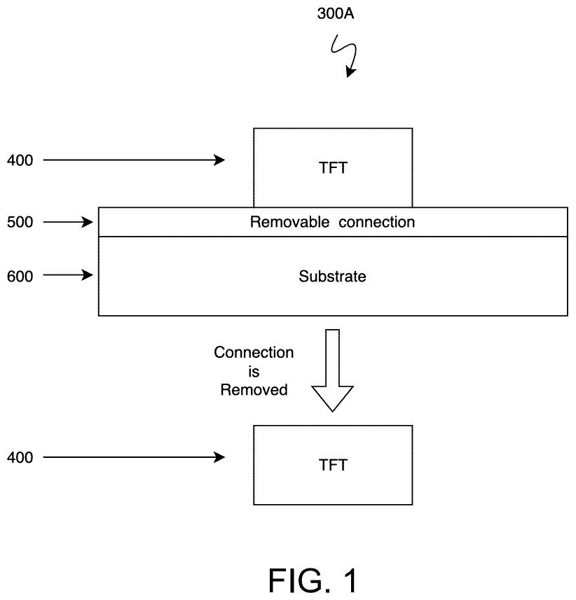

FIG. 1 depicts an illustration of the thin film transistor 400 fabrication for nanorobots. In this embodiment, the removable connection 500 is placed on the substrate 600 below the TFT 400. After this connection 500 is removed, the TFT 400 is separated from the substrate 600.

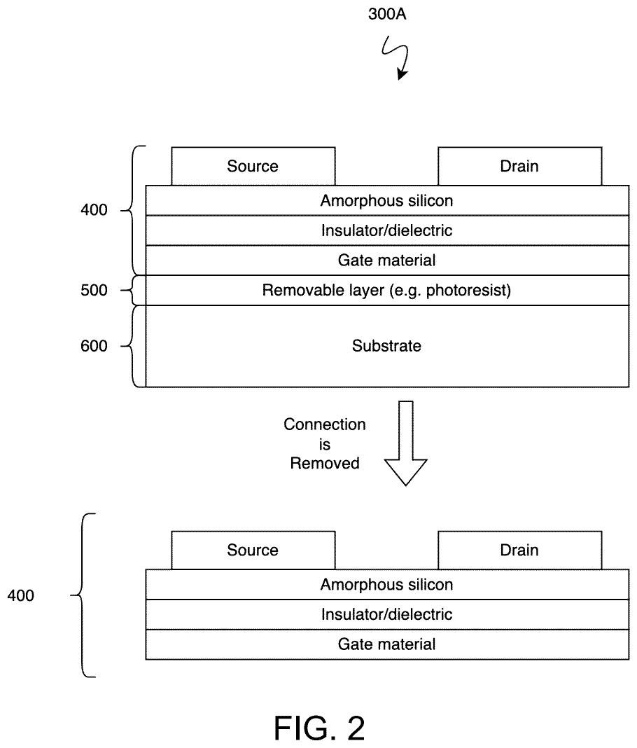

When the TFT 400 is disconnected from the substrate 600, the TFT 400 is alone. Fabricating the TFT 400 on a removable substrate can be done in several ways. The easiest way is to keep all else equal for the typical TFT fabrication process except to put a layer of photoresist between the substrate 600 and the first layer of the TFT 400A. This is depicted in FIG. 2. Once the TFT fabrication process is complete, additional steps are needed to remove the physical connection layer between the TFT and their substrate:
a. expose the photoresist to UV light (if it's positive photoresist)
b. use acid to remove the photoresist

FIG. 2 depicts a more in-depth fabrication of a single TFT using this detachment method. In this figure, a staggered bottom gate TFT 400 is shown with its normal fabrication technique, then with the additional removable layer 500, then finally when it is removed from the substrate 600. Although a staggered bottom gate TFT is used in this figure, any TFT design can be used.

The difference between the typical TFT electronics and this invention's TFT nanorobots is their size. TFT electronics typically have a surface area larger than a square millimeter. This is useful for their applications, but nanorobots need to be small. The nanorobots in this invention can be fabricated as smaller and disconnected TFT integrated circuits (IC).

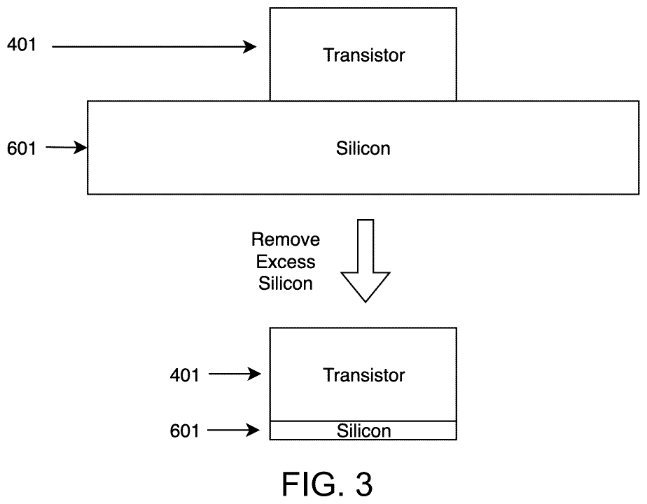

Other possible fabrication techniques are derivative of typical silicon manufacturing processes, such as manufacturing the nanorobots, then removing the excess silicon they were fabricated on. Techniques to remove the excess silicon are lasers, grinding, or sawing. This is shown in FIG. 3. The transistor 401 is fabricated on silicon substrate 601. Then the excess silicon is removed and transistor 401 is left connected to a smaller silicon substrate 601.

Because these nanorobots are going to be in a different environment than ICs typically see, there are some additional requirements or possibilities for these nanorobots. One of these is a sufficient coating. If these nanorobots are in water or any biological environment, they need to be waterproof. This coating can be applied before, during, or after the TFT fabrication process. There are a plethora of potential coatings for these nanorobots.

An embodiment of this invention uses polyethylene glycol, due to its biocompatibility and proven use in medical devices.

At this point in the nanorobot description, the nanorobots are flat. This greatly reduces their compute power and overall effectiveness. However, TFTs can be stacked. This can give us the desired 3D structure to have even more powerful and/or compute capable nanorobots.

FIG. 3 depicts an embodiment of this invention in which an outside device 700 transmits power and/or information to the nanorobot 100 inside the organism 800.

In an embodiment of this invention, the organism 800 is human.

There are a few possible embodiments of the wireless charging for the nanorobots in this invention, each suitable to their particular use case. In particular, these methods of powering the nanorobots will be useful:
a. inducing a current from a changing magnetic field
b. absorbing light with antennae
c. using photovoltaic power similar to photovoltaic cells
d. absorbing ultrasonic power with a piezoelectric energy harvester
e. chemical processes, such as a glucose biofuel cells

Living cells (including cancer cells and other pathogens) and viruses are heat sensitive; if they get too hot, they die. Using this fact, the nanorobots for this invention can be used to very precisely heat any cells, viruses, or regions that they are in. The advantage of nanorobots over quantum dots here is that nanorobots can decide whether or not they should heat.

To selectively heat the nanorobots, the power being received by the nanorobots needs to vary. Each method of powering the nanorobots results in a different method of selectively heating the nanorobots.

For the power system characterized by changing magnetic fields, the nanorobot can be powered by either creating an open circuit or a short circuit in the wire with the induced current. The nanorobot can decide when to open or short this circuit through control of a transistor or something similar.

For the power system characterized by receiving light with an antenna, there can be two separate frequencies of light (f_{A} and f_{B}). The nanorobot can operate normally with one frequency and receive excess power from the other frequency. This particular process can be done in several ways:
a. There can be two antennae and one antenna can be turned on or off to provide this additional light.
b. there can be a single antennae with the two frequencies of light providing different amounts of power. The nanorobot can change the shape of the antennae to change the frequency of light that it receives and thus the amount of power that it receives.

Another method of selectively heating the nanorobots that's independent to their power source is that the nanorobots can choose to receive their power in pulses by turning on and off their power reception mechanism according to whether or not the nanorobot determines it should be receiving power (or excess power).

It is useful for the nanorobots to enter the cells to determine whether or not the cell should be killed. The nanorobots are too large for diffusion through the cell membrane, so endocytosis is their only option to enter the cell. There are three main types of endocytosis: phagocytosis, pinocytosis, and receptor-mediated endocytosis. The nanorobots are too large for pinocytosis so that cannot be used here. Phagocytosis is mainly used by immune cells and can be used to target those. Nanorobots use receptor-mediated endocytosis to enter most cells, including most cancer cells.

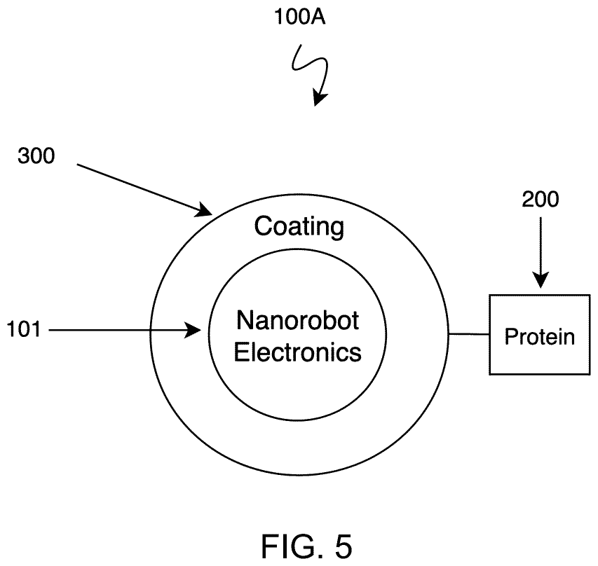

FIG. 5 depicts an embodiment that allows the nanorobot 100A to perform receptor-mediated endocytosis. To do this the nanorobots must have the appropriate proteins 200 on their surface. This can be any suitable protein 200 designed to target any suitable receptor. Attaching the protein 200 to the nanorobots 100A to facilitate this receptor-mediated endocytosis can be done in a number of ways, such as polyethylene glycol functionalization. Further details follow:
a. Coat 300 the surface of the nanorobot with polyethylene glycol (or any other suitable coating)
b. Attach a functional group, such as an amine or thiol to one end of the coating molecule.
c. Have this functional group react with the suitable protein 200. For ideal results, the suitable protein 200 should react at different parts when binding to the nanorobot 100 and when reacting with the cell receptor.

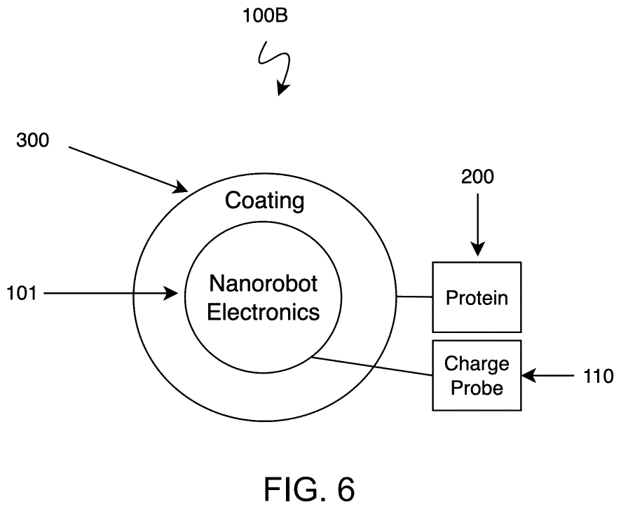

FIG. 6 depicts another embodiment of this invention, in which the proteins 200 on the surface of the nanorobots 100B can be used to assist with both endocytosis, exocytosis, phagocytosis, and apoptosis. It is desired to control when these processes occur and therefore when these proteins are properly expressed on the surface of the nanorobots through electrostatic inhibition and non-inhibition. To do this, a charge can be expressed near the protein. For example, this can be in the form of a probe 110 from the nanorobot 100B with an effective charge. If the charge is large enough and close enough the protein will not interact properly with the receptor used to perform endocytosis or exocytosis. This “nearby charge” mechanism can be used to control any other similar processes in which proteins are controlled through electrostatic inhibition/non-inhibition.

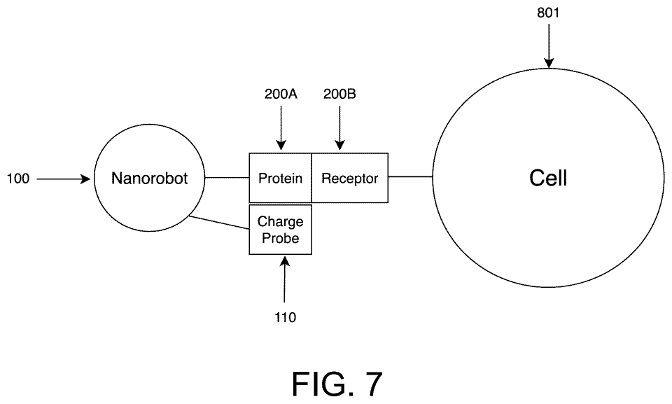

FIG. 7 depicts the interaction of the nanorobot 100 with the cell 801 in which the nanorobot's protein 200A is interacting with the cell's receptor 200B as a necessary step to endocytosis. The charge probe 110 is nearby, but not exhibiting a charge so as to allow for the protein 200A to behave uninhibited.

In an embodiment, enzymes that enter into lysosomes can gain admission by bearing mannose-6-phosphate. This protein 200 can be expressed on the surface of the nanorobots 100 when the nanorobot 100 should be expelled from the cell via exocytosis. As previously discussed, the nanorobot 100 can electrostatically inhibit/non-inhibit this protein 200 so that the nanorobot 100 can stay within the cell for a prolonged period of time.

To allow the nanorobot 100 to appear “foreign” to the immune system, the nanorobot 100 can produce charges on/near the surface of the nanorobot 100, such as through the charge probe 110. This can trigger any number of biological processes, including phagocytosis.

The nanorobots 100 can trigger phagocytosis by appearing like a target for the phagocyte and killing the cell once inside. The nanorobot can look like a target for the phagocyte in any number of ways, but specifically by attaching a protein 200 that the phagocyte is targeting or simply by presenting in any foreign way.

In another embodiment, the nanorobots 100 can control cell apoptosis by electrostatically inhibiting/non-inhibiting a cytotoxin or cytotoxic protein 200.

In another embodiment, the nanorobots can kill pathogens by emitting ionizing light.

Nanoelectromechanical (NEMS) and microelectromechanical (MEMS) can be used by the nanorobots 100. These NEMS/MEMS can change which proteins 200 are present on the surface, can change the orientation of the proteins 200, can move the charge probe 110, can puncture cell membranes, etc.

Other systems of movement, like using motor proteins, can be used to move the charge probe 110, the nanorobot 100, or do any process that requires actuation.

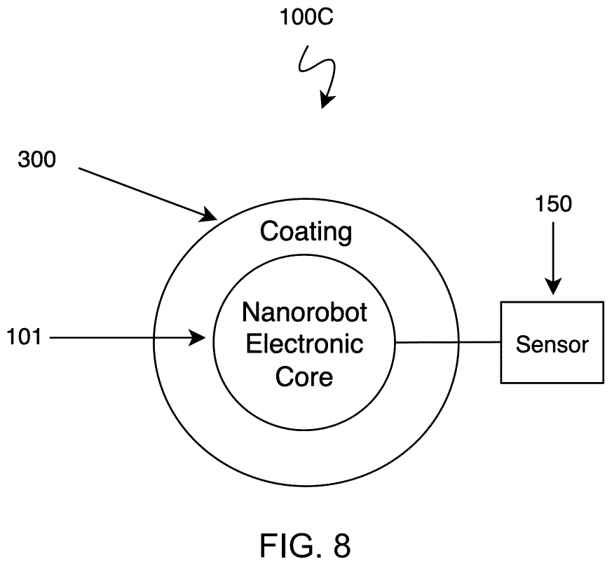

FIG. 8 depicts an embodiment in which the nanorobot 100C has a sensor 150. The nanorobots will be much more capable if they can sense their environment. This includes sensing environmental properties like pH, salinity, temperature. Additionally, in biological environments sensing specific chemicals and proteins is useful. This can be done with any number of sensors, but a particularly useful sensor for this application is nanowire field-effect transistors (FET). Nanowire field-effect transistors can be designed to sense specific chemicals and proteins.

In an embodiment, sensors 150 can detect lactate and oxygen concentrations to determine whether a cell is exhibiting the Warburg effect, which indicates the cell is cancerous.

In an embodiment, sensors 150 can detect leptin concentrations to determine whether or not a cell should be killed.

In an embodiment, sensors 150 can detect adiponectin concentrations to determine whether or not a cell should be killed.

In an embodiment, sensors 150 can detect proteins that indicate a cell is infected with a virus to determine whether or not a cell should be killed.

In an embodiment, sensors 150 can detect myxovirus resistance protein 1 which indicates a cell is infected with a virus to determine whether or not a cell should be killed.

In an embodiment, the nanorobot 100 can then act upon the sensed environment. It can perform a number of actions, but specifically the nanorobot can kill the cell it is in using any of the methods described herein.

In an embodiment, the sensors 150 can interact with an outside device 700 and determine that it should behave differently, such as being more aggressive with its cell killing.

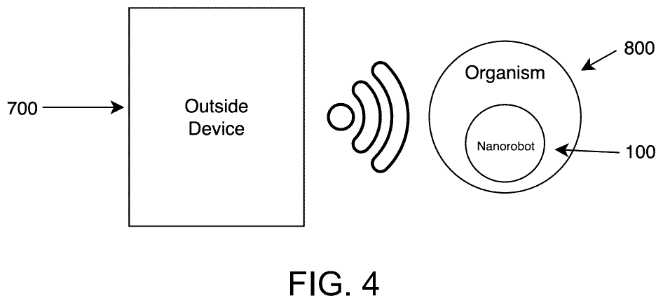

FIG. 4 depicts how nanorobots 100 can communicate with the outside device 700. Near field communication (NFC) uses the device's built-in clock. The nanorobots 100 might be too small to conveniently use a clock. Therefore, the nanorobots 100 require a new way of wireless communication.

In an embodiment, the nanorobot 100 interacts with an outside device 700 and a clock is induced. This can be through light, changes in electromagnetic fields, or through ultrasound.

RF-powered communication (also called harvest-and-communicate) is used today for small devices that have similar requirements to these nanorobots 100. This can be used to communicate with the nanorobots 100. Both reception and transmission are possible with RF-powered communication.

In an embodiment of this invention, the nanorobots 100 communicate synchronously when the outside device 700 induces the clock on the nanorobot with a modulating light, possibly even the light used to power the nanorobot 100.

Additional photodiodes, phototransistors, or light sensors 150 can also be used for communication. The nanorobot 100 can use the antenna for power with these additional components at any wavelength using standard communication protocols. These additional devices can be used for transmission and reception of data.

In an embodiment of this invention, the nanorobots assist in or control the release of drugs to allow for targeted drug release, using any number of drug release mechanisms.

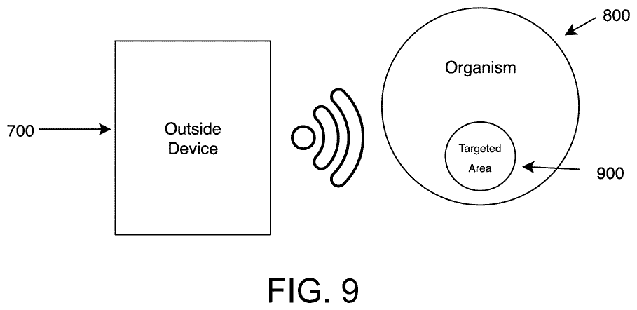

FIG. 9 depicts an embodiment of the invention in which the outside device 700 specifies a targeted area 900 of the organism 800 in which the nanorobot behaves differently, such as being more aggressive, releases drugs, or any number of behaviors obvious to those experienced in the state of the art.

In an embodiment of this invention, the targeted area 900 can be determined by radiating from a point source, radiating from a linear source, radiating from a planar source, following a linear path such as those from lasers, creating a standing wave, creating a slowly moving wave, creating a pattern from the superposition of multiple outside devices 700, or any other method of creating a standing wave obvious to those experienced in the state of the art.

What is claimed is:

1. A system for nanorobots comprising:
a fabrication method;
a power generator;
electrically controlled proteins;
sensors; and
a method of killing an organism's cells;
wherein a nanorobot is fabricated, put into the organism, powered, uses sensors to determine whether to kill the organism's cells, and selectively kills the organism's cells.

2. The system for nanorobots of claim 1, wherein the fabrication method uses thin film transistors detached from their substrate.

3. The system for nanorobots of claim 1, wherein the fabrication method fabricates the nanorobot on a silicon wafer and removes the excess silicon.

4. The system for nanorobots of claim 1, wherein the power generator absorbs energy emitted from an outside device.

5. The system for nanorobots of claim 4, wherein the power generator absorbs ultrasound energy generated from an outside device.

6. The system for nanorobots of claim 4, wherein the power generator absorbs light energy emitted from an outside device.

7. The system for nanorobots of claim 4, wherein the power generator absorbs energy in the form of changing electromagnetic fields generated from an outside device.

8. The system for nanorobots of claim 1, wherein the power generator uses chemical processes to produce electric power.

9. The system for nanorobots of claim 1, wherein the electrically controlled proteins are functionally disabled through controlled electrostatic inhibition.

10. The system for nanorobots of claim 9, wherein the electrically controlled proteins are used for endocytosis.

11. The system for nanorobots of claim 9, wherein the electrically controlled proteins are used for exocytosis.

12. The system for nanorobots of claim 9, wherein the electrically controlled proteins are used for apoptosis.

13. The system for nanorobots of claim 9, wherein the electrically controlled proteins are used for phagocytosis.

14. The system for nanorobots of claim 1, wherein the sensors are capable of detecting chemical properties of the nanorobot's surroundings.

15. The system for nanorobots of claim 14, wherein the sensors are capable of detecting chemicals of the nanorobot's surroundings.

16. The system for nanorobots of claim 15, wherein the sensors are capable of detecting chemicals of the nanorobot's surroundings using nanowire field-effect transistors.

17. The system for nanorobots of claim 1, wherein the method of killing the organism's cells is performed by heating the nanorobot.

18. The system for nanorobots of claim 1, wherein the method of killing the organism's cells is performed by emitting ionizing radiation.

19. A system for nanorobots comprising:
a fabrication method;
a power generator;
electrically controlled proteins;
sensors;
a method of killing an organism's cells; and
a method of communicating with the nanorobots with an outside device;
wherein a nanorobot is fabricated, put into the organism, powered, uses sensors to determine whether to kill the organism's cells, selectively kills the organism's cells, and the nanorobots can communicate with an outside device.

20. A system for nanorobots comprising:
a fabrication method;
a power generator; and
a method of drug release;
wherein a nanorobot is fabricated, put into the organism, powered, and the nanorobots can release drugs.
